(19) United States
(12) Patent Application Publication
(10) Pub. No.: US 2012/0132199 A1
(43) Pub. Date: May 31, 2012

(54) ATOMIZER

(75) Inventor: Lothar Kiesewetter, Berlin (DE)

(73) Assignee: BOEHRINGER INGELHEIM INTERNATIONAL GMBH, Ingelheim am Rhein (DE)

(21) Appl. No.: 13/201,744

(22) PCT Filed: Feb. 10, 2010
(86) PCT No.: PCT/EP10/00796

§ 371 Date: Oct. 5, 2011

(30) Foreign Application Priority Data
Feb. 18, 2009 (EP) 09002253.4

(51) Int. Cl. A61M 11/00

(52) U.S. Cl. 128/200.22; 128/200.14

(57) ABSTRACT

A nebuliser of simple construction is proposed for nebulising a medicament preparation for medicinal aerosol therapy. The medicament preparation is accommodated free of residual gas in a lobe-shaped bag consisting of two annular film pieces. A conveying pump is pneumatically driven and is sealed by a hose seal.

The present invention relates to a nebuliser according to the preamble of claim 1, 5 or 13 as well as to a method for nebulising a liquid, in particular a medicament preparation.

The present invention relates in particular to a nebuliser with a piston pump for the highly accurate dosing a medicament preparation or the like, and its subsequent nebulising to form a very fine spray mist.

The starting point of the present invention is a nebuliser in the form of an inhaler, as is described in WO 2006/125577 A2. The nebuliser comprises as reservoir for a medicament preparation to be nebulised a chargeable, rigid container with an inner bag containing the medicament preparation and a conveying device with a piston and an associated drive spring for conveying and nebulising the medicament preparation. With the known nebuliser the drive spring has to be manually tensioned in each case before nebulising can take place.

The known nebuliser is composed of relatively many parts and requires very tight manufacturing tolerances.

The object of the present invention is to provide a nebuliser as well as a method for nebulising an in particular medicinal liquid, whereby a simple, inexpensive and/or robust construction and/or a simple operation are made possible.

The above object is achieved by a nebuliser according to claim 1, 5 or 13 or by a method according to claim 15. Advantageous modifications are the subject of the subclaims.

According to a first aspect of the present invention a conveying device of the nebuliser comprises a piston displaceable in a cylinder and an associated roll-type or hose-type seal for producing a seal between the cylinder and piston. This allows a very simple, inexpensive and in particular hermetic structure, in which frictional forces can be significantly reduced compared to the normally used annular seal or the like. This facilitates the operation and the drive.

According to a second aspect of the present invention the conveying device comprises a pump that can be driven by a hydraulic or pneumatic working medium, in order to subject the in particular medicinal liquid to pressure and to nebulise and/or dispense the liquid for example as a jet or in some other way through a discharge nozzle. This permits a very simple operation. In particular a manual tensioning of a drive spring or the like is no longer necessary.

According to a third aspect of the present invention the conveying device can be driven with a hydraulic or pneumatic working medium and comprises a pressure booster in order to increase the pressure acting on the liquid compared to the pressure of the working medium. For this purpose in particular a double or step piston is used, which at the same time preferably forms a pump piston for the liquid. This allows a very simple, inexpensive and/or compact structure, in which very high dispensing pressures and thereby in particular a very good nebulising can be achieved also with not too high operating pressures of the working medium.

According to a fourth aspect of the present invention the conveying device comprises a pressure container with a hydraulic or pneumatic working medium, in order to dispense the in particular medicinal liquid under pressure and in a desired manner, in particular through a discharge nozzle, wherein the pressure container is formed so as to be at least partially spring-elastic—for example by means of a corrugated wall—so as to reduce the pressure drop of the working medium under pressure in particular when the nebuliser is subjected to multiple use. This allows a particularly compact and correspondingly simple and inexpensive as well as robust construction of the nebuliser.

According to a fifth aspect of the present invention the reservoir is formed from a bag of two film pieces joined to one another in a surrounding or annular joining region and/or the bag is formed lobe-shaped, annular-shaped or disc-shaped. This allows a particularly simple and inexpensive construction. Particularly preferably the reservoir consists exclusively of the bag. The bag can be produced very simply with flat, surrounding and/or annular joins, in particular by welding, sealing, bonding or the like. In particular gussets are avoided in the bag, which can cause problems as regards sealing.

According to a sixth aspect of the present invention the nebuliser or a reservoir, such as the bag, comprises a non-return valve that opens for the dispensing of the liquid in the delivery direction and automatically closes in the opposite direction, wherein the non-return valve can open magnetically or electrically or mechanically to allow liquid to flow in the direction opposite to the delivery direction, in particular to fill the reservoir. This allows a very simple construction and a very simple filling of the nebuliser and reservoir. Such a reservoir can also be realised and charged independently of the nebuliser.

The individual aspects and other features of the present invention can be implemented independently of one another and/or can be combined with one another in any desired combination.

Further advantages, features, properties and aspects of the present invention follow from the claims and the following description of a preferred embodiment given with the aid of the drawings, in which:

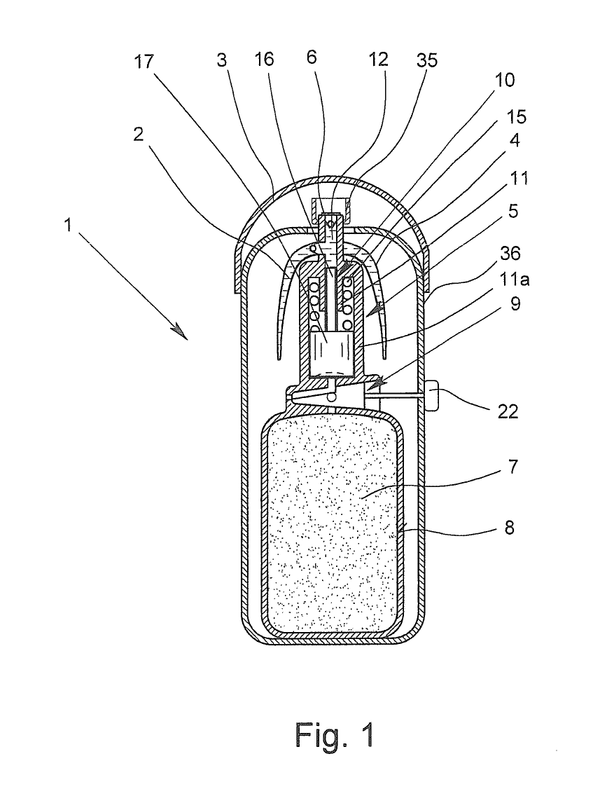

FIG. 1 is a schematic section of a proposed nebuliser with a closed cap;

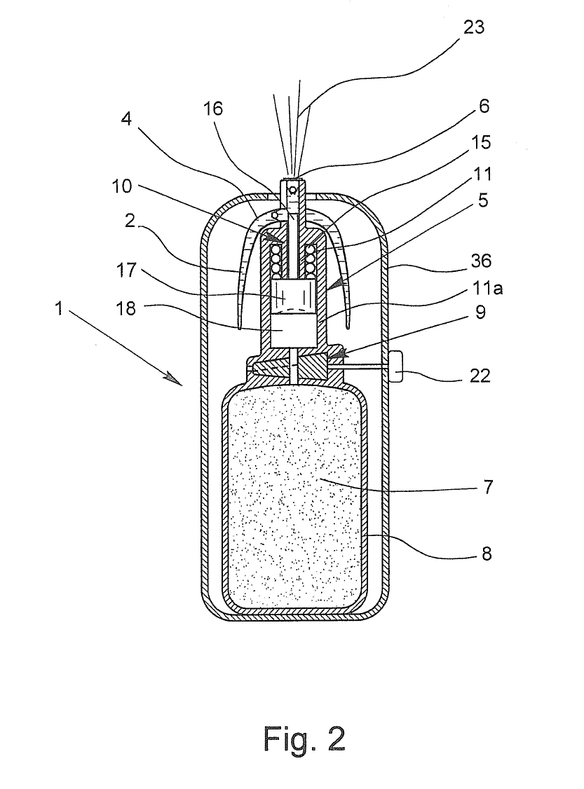

FIG. 2 is a schematic section of the nebuliser without the cap, during the nebulising;

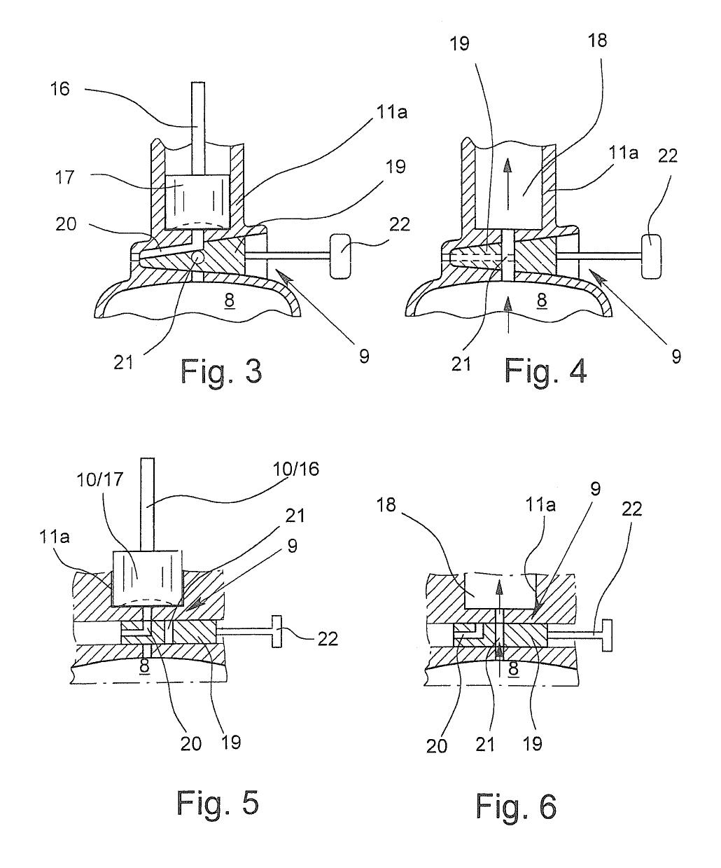

FIG. 3 is a schematic sectional enlargement of a control valve of the nebuliser in a non-actuated state;

FIG. 4 is a representation corresponding to FIG. 3 of the control valve in the actuated state for the nebulising;

FIG. 5 is a representation corresponding to FIG. 3 of the control valve according to a variant in the non-actuated state;

FIG. 6 is a representation corresponding to FIG. 4 of the control valve according to the variant according to FIG. 5, but in the actuated state for the nebulising;

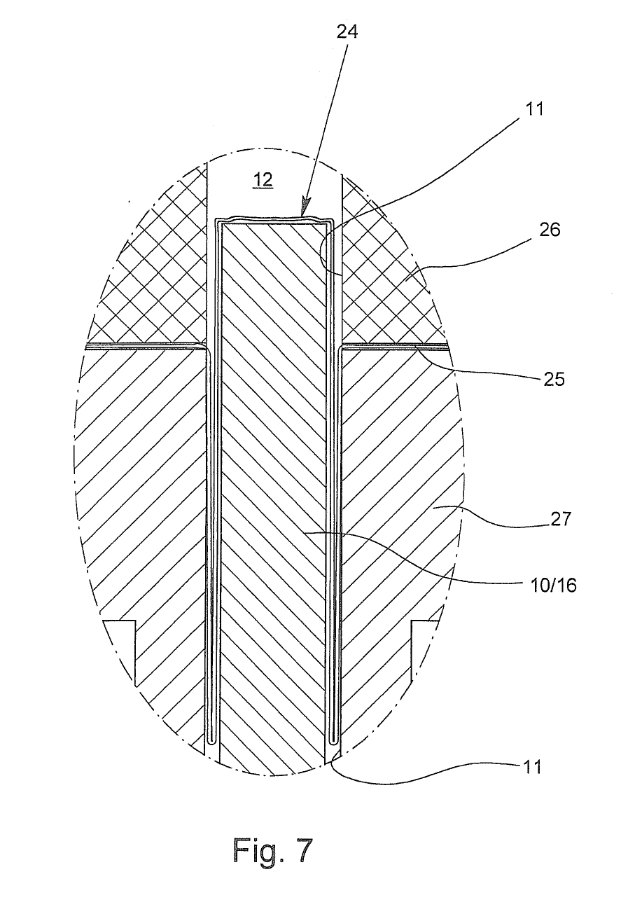

FIG. 7 is a schematic section of the nebuliser in the region of a piston with a roll-type or hose-type seal;

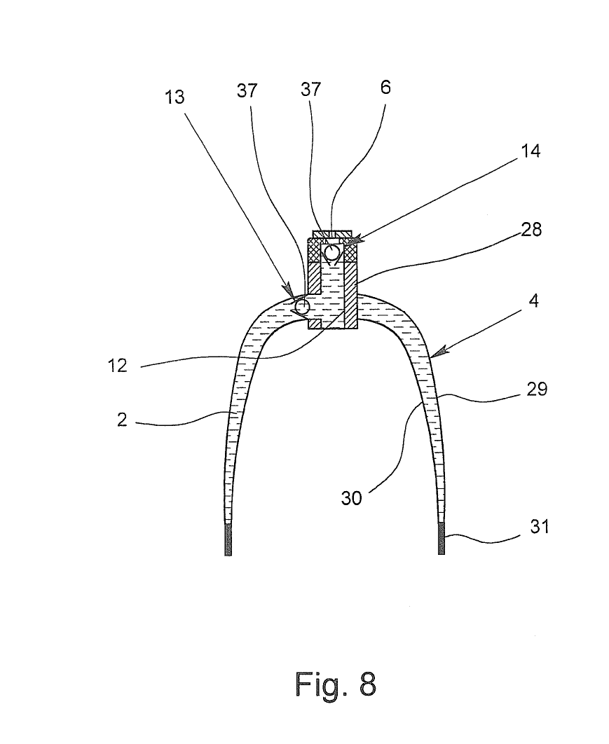

FIG. 8 is a schematic section of a reservoir of the nebuliser in the installed state with a connecting and dispensing piece;

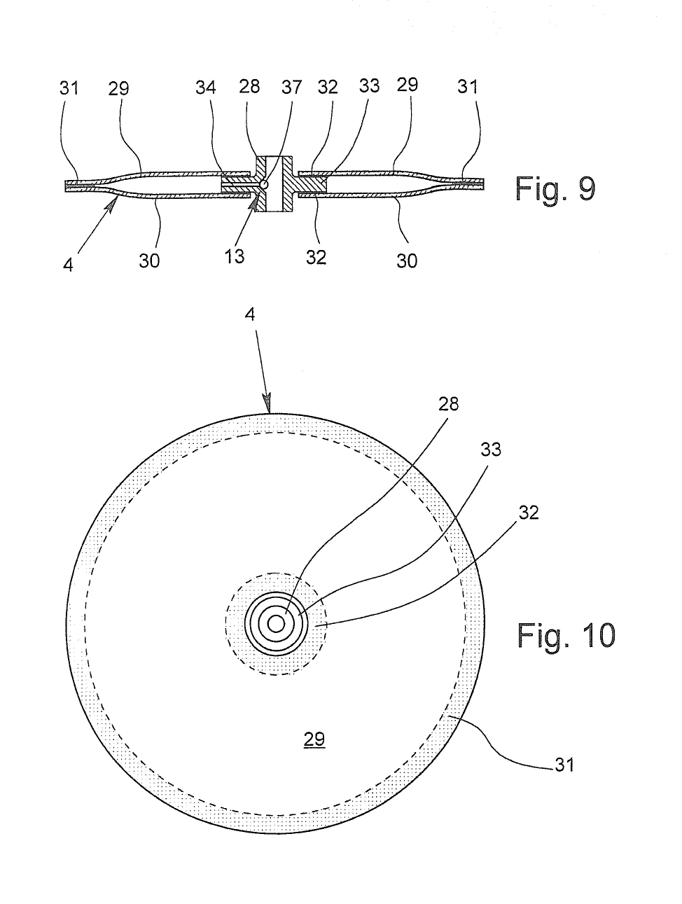

FIG. 9 is a schematic section through the reservoir in the unfilled state in order to illustrate the manufacture;

FIG. 10 is a plan view of the reservoir according to FIG. 9; and

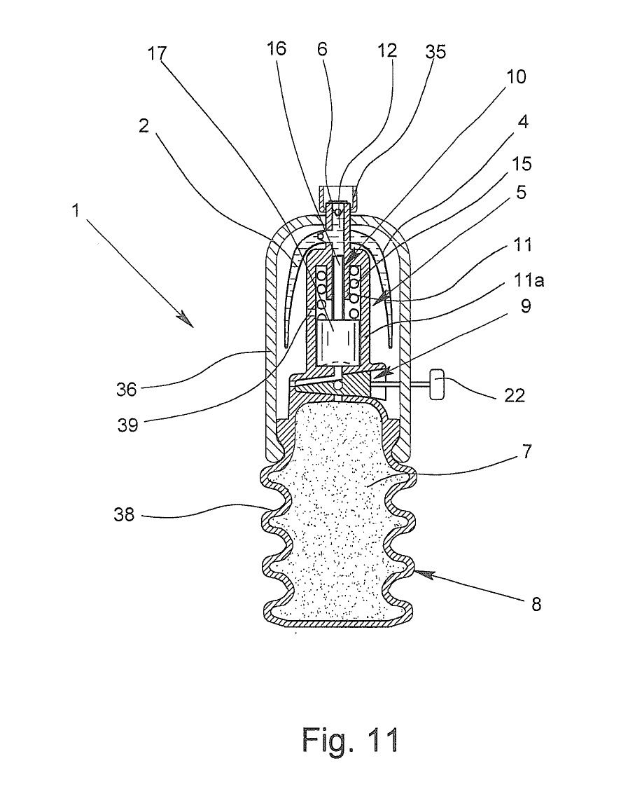

FIG. 11 is a schematic section of the nebuliser according to a further variant.

In the Figures the same reference numerals are used for identical or similar parts in which corresponding of comparable properties and advantages are achieved, although a repeated description is omitted.

FIG. 1 shows in a schematic section a proposed nebuliser 1 for nebulising a liquid, in particular a medicament preparation 2. In particular the nebuliser 1 is an inhaler for a medicinal or therapeutic aerosol treatment. The term “liquid” is however also preferably understood to cover other liquids, dispersions, mixtures of solutions and dispersions or the like.

FIG. 1 shows the nebuliser 1 in the non-actuated or closed state, in particular with a cap 3. FIG. 2 shows the nebuliser 1 in a schematic section corresponding to FIG. 1, but without the cap 3 and in the actuated state and during the nebulising and dispensing.

The nebuliser 1 has a reservoir for the liquid to be dispensed, which in this case is preferably formed by a bag 4. The preferred structure of the bag 4 will be described in more detail later.

The nebuliser 1 preferably comprises a conveying device 5 in order to pressurise the liquid 2, in particular in individual doses, and to nebulise or dispense it in some other way—for example as a jet—through a discharge nozzle 6 of the nebuliser 1. The discharge nozzle 6 is shown only schematically.

The conveying device 5 can preferably be driven by a hydraulic or pneumatic working medium 7. In particular the conveying device 5 comprises a pump, particularly preferably a metering and/or piston pump, or forms such a pump. This can preferably be driven by the working medium 7, in particular so as to pressurise the liquid and nebulise or dispense it in some other way through the discharge nozzle 6.

Preferably the nebuliser 1 comprises a pressure container 8 for or with the working medium 7. Particularly preferably the pressure container 8 is formed as an insertable and/or connectable, for example screw-on, cartridge. The working medium 7 is preferably air or another gas such as carbon dioxide.

According to an also independently realisable aspect of the present invention the pressure container 8 and its wall can preferably be pretensioned and/or at least partially elastically deformed so as to increase its volume. This will be also explained later by way of example with the aid of FIG. 11.

The pressure container 8 can be connected in a gas-tight manner by means of a suitable connection to the conveying device 5 and/or to an intermediately connected control valve 9 of the nebuliser 1.

The conveying device 5 and pump comprise a piston 10 and a pump section 16, so as to aspirate, pump, meter and/or pressurise the liquid to be dispensed and nebulise and/or dispense the liquid through the discharge nozzle 6. The piston 10 and its working section 17 can preferably be driven by the working medium. However, in principle some other drive can also be provided.

The piston 10 and its pump section 16 can preferably be displaced and/or guided in a cylinder 11. In this connection it should be noted that the piston 10 and its pump section 16 are preferably cylindrical in shape and the cylinder 11 is correspondingly formed as a hollow cylinder; however, in principle other mutually adapted cross-sectional shapes are also possible.

Due to the reciprocatory movement of the piston 10 and pump section 16 in the cylinder 11 liquid can alternately be aspirated (suction stroke) from the reservoir and bag 4 into a pump space 12 and then dispensed through the discharge nozzle 6 (pressure or nebulising stroke).

The nebuliser 1, the reservoir and the bag 4 or the conveying device 5 preferably comprise an inlet valve or first non-return valve 13, as is illustrated in the section according to FIG. 8, in order to connect the reservoir and the bag 4 in a fluid manner to the pump space 12, so that during the suction stroke liquid can be aspirated from the bag 4 into the pump space 12, while during the pressure and nebulising stroke however no liquid is conveyed back into the bag 4.

The nebuliser 1 and the conveying device 5 furthermore preferably comprise an outlet valve or second non-return valve 14, which is arranged between the pump space 12 and the discharge nozzle 6 so as to prevent aspiration of air through the discharge valve 6 into the pump space 12 during the suction stroke and to allow a dispensing of the liquid from the pump space 12 through the discharge nozzle 6 in the pressure and nebulising stroke.

In the suction stroke the inlet valve or first non-return valve 13 opens and closes the outlet and second non-return valve 14. In the pressure and nebulising stroke the inlet or first non-return valve 13 closes and opens the outlet or second non-return valve 14.

The pump space 12 is in the illustrated Example formed and bounded substantially by the piston 10 and the cylinder 11 and possibly the valves 13 and 14.

The (lower) position of the piston 10 shown in FIG. 1 after the suction stroke, i.e. before the pressure and nebulising stroke, is termed the initial position in the present invention. The (upper) position of the piston illustrated in FIG. 2 after completion of the pressure and nebulising stroke and before the subsequent suction stroke is termed the end position in the present invention. The stroke between the two positions defines the amount and dose of liquid dispensed each time.

The nebuliser 1 and the conveying device 5 or pump preferably comprises a restoring means, in particular a spring 15, which is associated with the piston 10 in particular for returning it to the initial position. In particular the suction stroke and the aspiration of liquid from the reservoir or bag 4 takes place through the force of the restoring means and spring 15. However, other structural solutions are also possible.

The nebuliser 1 and the conveying device 5 preferably comprise a pressure booster in order to raise the pressure acting on the liquid compared to the pressure of the working medium 7. The pressure booster is in particular formed by a double and/or step piston, which here is preferably formed by the piston 10. The step piston preferably also forms at the same time the pump piston acting directly on the liquid. However, other structural solutions are also possible. In particular the pressure booster can also be formed independently and mechanically separated from the pump, the pump piston or the like, and for example can therefore act on an intermediate medium, and/or via a drive on the pump and the pump piston.

In the illustrated Example the piston 10 is formed as a double piston, which on the one hand can be driven by the working medium 7 and on the other hand pumps the liquid. In this connection the working medium 7 acts on a larger effective cross-section of the piston 10, whereas the piston 10 in contrast presses with a correspondingly smaller cross-section on the liquid in order to achieve the desired pressure increase.

Particularly preferably the step piston or piston 10 comprises a relatively thin pump section 16, which is associated with and engages in the pump space 12, and the working section 17 preferably substantially enlarged in cross-section compared to the pump section 16, which is movable in and is guided in a particularly tight manner in a cylinder 11a. For example the working section 17 has a diameter of about 5 mm and the pump section 16 has a diameter of less than 2 mm. The stroke path of the piston 10 is preferably about 10 mm.

Particularly preferably the piston 10, in particular a double or step piston, is formed in one piece. It preferably consists of metal, ceramics or another suitable material or composite material.

In the illustrated Example the spring 15 is preferably supported at one end on a shoulder at the transition from the working section 17 to the pump section 16. The spring 15 engages with its other end for example in an annular section that surrounds the cylinder 11 on the outside. However, other structural solutions are also possible here.

A working space 18 is associated with the working section 17, as shown in FIG. 2, which through the working medium 7 can be charged in order to act on the working section 17 and the pressure booster or the piston 10, in particular so as to drive the latter.

The charging of the working space 18 and/or working section 17 by the working medium 7 is controlled by the control valve 9.

FIG. 3 shows very schematically in a sectional representation of the nebuliser 1 the control valve 9 in the non-actuated state. In this state the working space 18 is ventilated to the outside. This is effected in the illustrated Example by a corresponding valve element 19, which is provided with a ventilation channel 20 that connects the working space 18 to the surroundings.

FIG. 4 shows in a representation corresponding to FIG. 3 the control valve 9 in the actuated position. Here a connecting channel 21 of the valve element 19 formed in particular by a through hole connects the pressure container 8 to the working space 18, so that the working section 17 and piston 10 are pressurised and driven by the working medium 7. The arrows in FIG. 4 indicate the flow of the working medium 7.

In order to actuate and adjust the valve element 19 the control valve 9 preferably has an associated handle 22 or some other adjusting element.

The control valve 9 may for example be formed so that it remains in the actuated state only during an actuation of the handle 22 or for a specified time or only until the piston 10 has reached the end position. However, the control valve 9 can also be formed so that it can be adjusted from the actuated state to the non-actuated state and vice versa, in each case only by actuating the handle 22 or the like.

In the Example illustrated in FIGS. 3 and 4 the control valve 9 is preferably formed as a turning valve or rounded cock valve. FIGS. 5 and 6 show in representations corresponding to FIGS. 3 and 4 a variant of the control valve 9 in the form of a slide valve. The function is the same in this case.

The handle 22 of the control valve 9 has—preferably pretensioned from plastics parts—in particular two stable settings, namely on the one hand the non-actuated position (FIGS. 3 and 5) and the actuated position (FIGS. 4 and 6). The handle 22 or another operating element is preferably formed as a pivoted lever or press button. Depending on requirements it is possible to provide for a switchover between the handle 22 and valve element 19 and/or to provide the control valve 9 with a lock, for example to prevent unintentional operation or the like.

The control valve 9 is preferably formed so that—for example by means of a rotation or translation spring, not shown—it always returns to the stationary or non-actuated state (FIGS. 3 and 5).

Starting from the state of the nebuliser 1 shown in FIG. 1 (the piston 10 is in the initial position), the control valve 9 can be actuated after the cap 3 has been opened and removed (if necessary a coupling or blocking of the control valve 9 is provided, so that when the cap 3 is closed no actuation of the control valve 9 and thus no dispensing of liquid 2 is possible). Due to the actuation of the control valve 9 and thus of the nebuliser 1 the piston 10 is driven by the working medium 7 and the liquid contained in the pump space 12 is subjected to pressure and is dispensed, in particular nebulised, through the (optional) second non-return valve 14 and the discharge nozzle 6, until the piston 10 reaches its end position.

When the control valve 9 then returns or is restored to its non-actuated state, the working space 18 is ventilated and the piston 10 can in particular return to its initial position only through the force of the restoring means (spring 15) under aspiration of liquid into the pump space 12. Accordingly, a very simple, user-friendly operation is achieved.

The bag 4 collaborates in the discharge of liquid. This can be accomplished without any problem and virtually without any pressure loss on account of its flexible design. Accordingly, the liquid can be aspirated very easily from the reservoir into the pump space 12, so that the force of the spring does not have to be very large. This is compatible with the nebulising, since the spring force has to be overcome in the pressure and nebulising stroke.

Preferably the spring 15 is installed pretensioned in order to ensure that the piston 10 always completely returns to the initial position.

In order to achieve as uniform a nebulising as possible, a pressure control or regulation can be provided for the working medium 7. In particular a pressure control or regulating valve can be provided if necessary in order to maintain the working pressure of the working medium 7 on the piston 10 and the pressure booster as constant as possible. Particularly preferably the control valve 9 is then formed directly as a pressure control or regulating valve. However, other structural solutions are also possible.

In addition or alternatively the nebuliser 1 and the control valve 9 can also comprise a spray timing adjustment. This can be achieved for example by using a discharge nozzle 6 with a corresponding flow resistance, or by employing a throttle or the like.

The pressure of the working medium 7 in the pressure container 8 is preferably about 1 to 5 MPa, in particular about 2 to 3 MPa.

The pressure booster is preferably designed to raise the pressure by at least a factor of 5, in particular by a factor of 10 or more.

The nebuliser 1 is preferably formed so that during the dispensing, the liquid in the conveying device 5 and in the pump chamber 12 reaches a pressure of substantially 5 MPa to 60 MPa, in particular about 10 MPa to 50 MPa, due to the force of the working medium 7. Particularly preferably in the dispensing and nebulising of the liquid a pressure of about 5 to 60 MPa, in particular about 10 to 30 MPa, is reached at the discharge nozzle 6 and at its nozzle opening(s). The liquid is then converted into a spray mist 23 (aerosol), as illustrated schematically in FIG. 2.

The droplets of the spray mist 23 have an aerodynamic diameter of preferably at most 20 μm, preferably about 3 μm to 10 μm.

If necessary the nebulising action and the nebulising effect can be achieved or further assisted by dispensing a plurality of jets from the discharge nozzle 6, which intermingle immediately after leaving the discharge nozzle 6. Alternatively or in addition a deceleration of the exit velocity and/or propagation velocity of the spray mist 23 can also be achieved in this way.

The amount of liquid dispensed per stroke or dose is preferably about 10 μl to 50 μm, most preferably about 15 μm.

The discharge duration per pressure and nebulising stroke is preferably 1 to 2 seconds or about 1 to 2 inspirations of a user (not shown).

FIG. 7 shows in a very schematic section a region of the conveying device 5 and pump, and more specifically a preferred sealing of the piston 10 with respect to the cylinder 11. Preferably a roll or hose seal 24 is associated with the piston 10 to form the seal between the cylinder 11 and piston 10. The seal 24 preferably consists of a film-like hose or some other suitable piece of material, which hermetically seals the pump space 12 at its end and is reversibly deformed by the piston 10 as a result of its reciprocatory movement. In particular the seal 24 rolls on and off on the piston 10 and/or in the cylinder 11 during the movement of the piston. Thus, a very small degree of friction can be achieved with a very small dead volume and with a hermetic and tight seal between the piston 10 and cylinder 11. Also, a very simple construction is achieved.

If necessary the seal 24 can also be formed in one piece with the bag 4 or with a film piece of the bag 4 and/or can be connected thereto.

The seal 24 is formed from a preferably very soft and/or gas-type material.

In the illustrated Example the seal 24 is held in a clamping manner and/or tightly with an end region 25, for example, between the cylinder structural parts 26 and 27 forming the cylinder 11. However, other structural solutions are also possible.

The roll or hose seal 24 is formed particularly preferably by a sealing hose, which during the installation is drawn over the piston 10 and in the partly folded-back function region is formed with its preferably flange-like edge region 25 as a sealing disc and/or is inserted or clamped in the cylinder 11.

The annular gap between the piston 10 and cylinder wall preferably corresponds substantially to double the wall thickness of the hose material. During the reciprocatory movement of the piston 10 the hose correspondingly rolls off.

The aforementioned roll or hose seal 24 can alternatively or in addition also be used for the seal against the working medium 7, i.e. on the drive side in the working space 18 and for the working section 17. The proposed roll or hose seal 24 serves here particularly preferably at the same time as a seal for the working space 18 and to seal the working medium 7 against the liquid.

The nebuliser 1 has minimal frictional losses in use. The roll or hose seal 24 has a substantially lower friction than conventional sliding friction piston seals. Furthermore the manufacturing tolerance is substantially less, since for example structural parts with high surface quality are not necessary and no special matching of the piston 10 in the cylinder is required.

The proposed roll or hose seal 24 can if necessary also be replaced by a bellows-type membrane or the like.

The use of the roll or hose seal 24 for the liquid and/or the working medium 7 is preferably also independent of other aspects of the present invention, and in particular can also be used with other nebulisers or inhalers or other dispensing devices.

A nebulising of the liquid can be carried out in a particularly simple manner as proposed above. After opening the nebuliser 1 and the cap 3, the control valve 9 is actuated. The working medium 7 charges the piston 10 so that the piston 10 moves against the force of the spring 15 to the end position and the liquid contained in the pump space 12 is pressurised and dispensed and nebulised through the discharge nozzle 6. Liquid is then aspirated from the reservoir—in this case the bag 4—by the piston 10 into the pump space 12. The piston 10 is thereby restored again to the initial position due to the force of the spring 15, after the control valve 9 has returned or restored to its initial position or ventilation position or its non-actuated state, i.e. when the working space 18 is ventilated, so that the pressure on the piston 10 and working section 17 is reduced at least to a sufficient extent or is completely cancelled.

The nebuliser 1 can if desired be closed again with the piston 10 in the initial position or the end position and stored. If the nebuliser 1 is to be stored with the piston 10 in the end position also for a fairly long period of time, then a locking device or the like (not shown) can be provided in order to be able to mechanically lock the piston 10 in the end position. This lock can be cancelled for example by opening the cap 3.

Alternatively or in addition it is also possible for the cap 3 to control, actuate and/or block the control valve 9. For example, the cap 3 can be coupled in such a way to the control valve 9 or other device so that on opening the cap 3 the control valve 9 switches to the non-actuated state and the ventilation position—at least for a short period—and ventilates the working space 18. This would be advisable if the nebuliser 1 can be stored with the piston 10 in the end position with the cap closed.

The cap 3 can for example also be opened in several stages however, and at the same time serve or operate as an actuating element or handle 22. Thus, for example, the actuation of the control valve 9 can be triggered if the cap 3 reaches its opened end position or is opened somewhat further from its normal end position against the spring force, so as to allow the working medium 7 to flow into the working space 18 and effect the nebulising.

Furthermore, it is also possible for the cap 3 and/or another part of the nebuliser or housing to cover or block the control valve 9 or its handle 22 in the closed state so that the control valve 9 or the handle 22 can be actuated only when the nebuliser 1 is opened.

As already mentioned, the reservoir for the liquid is particularly preferably formed as a bag 4. FIG. 8 shows in a very schematic section of FIGS. 1 and 2 the bag 4 with an associated connecting piece 28, in which preferably the first non-return valve 13 or some other inlet valve is integrated, wherein the second non-return valve 14 or some other outlet valve and the discharge nozzle 6 in the illustration according to FIG. 8 are installed or mounted on the connecting piece 28. The bag 4 is preferably formed so as to be flexible or deformable, and in particular is very soft.

FIG. 8 shows the bag 4 with a shape that in cross-section is bent approximately in the shape of a parabola, which it can adopt for example in the nebuliser 1. However, the bag 4 can in principle also adopt any other shape. FIG. 9 shows in a schematic section the bag 4 with associated connecting piece 28 in the unfilled state, for example during or immediately after the production. FIG. 10 shows a plan view of the bag 4 according to FIG. 9.

The bag 4 is preferably formed from two film pieces 29,30, which in particular are joined to one another only in a surrounding or annular and/or external joining region 31. Particularly preferably the film pieces 29, 30 are joined to one another by bonding, sealing, in particular heat sealing, or welding, in particular ultrasound welding.

The joining region 31 is preferably formed at least substantially flat The film pieces 29, 30 are preferably joined to one another only on their insides.

Particularly preferably the film pieces 29, 30 are not bent over in the transition to the joining region 31. Instead, the film pieces 29, 30 transform preferably at least substantially flat or smoothly in the joining region 31; this applies in particular also in the installed state.

The film pieces 29, 30 are in each case preferably formed at least substantially disc-shaped or annular-shaped and/or in one piece (in particular seamless). In particular in each case they form a closed ring. This minimises necessary welded joints or other joins.

Preferably the bag 4 and its flexible wall is formed only from the two film pieces 29, 30.

The bag 4 is preferably formed lobe-shaped, annular-shaped or disc-shaped and/or flat and/or without gussets.

Preferably the connecting piece 28 is joined directly to the bag 4 and the film pieces 29, 30. Preferably the connecting piece 28 has for this purpose a joining region 32 that is formed in particular all the way round and/or annular-shaped and/or at least substantially flat.

The film pieces 29, 30 are joined to one another preferably by bonding or welding, in particular laser welding or ultrasound welding.

The film pieces 29, 30 are joined to the joining region 32 preferably by bonding or welding, in particular ultrasound welding.

Preferably the connecting piece 28 has two separate joining regions 32, which are joined or can be joined to two different film pieces 29, 30 or sides of the bag 4. In particular these joining regions 32 are arranged on opposite sides of the connecting piece 28 and/or coaxially or above one another. In particular the joining regions 32 preferably face away from one another.

Particularly preferably the connecting piece 28 comprises a flange, in particular an annular flange 33, for the formation of a joining region 32 or the two joining regions 32 for the bag 4, as illustrated in FIG. 9. The joining regions 32 are then preferably formed on the front or axial sides of the flange. The joining regions 32 are preferably both formed annularly or are closed, in order to permit and ensure a simple, continuous and surrounding or closed seal and connection to the film pieces 29, 30.

The connecting piece 28 and the annular flange 33 are preferably inserted in an at least substantially flat surface region of a film piece 29 or 30 or of both film pieces 29, 30.

Preferably the connecting piece 28 comprises a connecting channel 34 for the fluid connection of the bag 4, The connecting channel 34 extends preferably radially through the flange 33. For this purpose the flange 33 can if necessary be thickened in the region of the connecting channel 34 or have a sufficient thickness all the way round.

In FIG. 9 the flange 33 is shown having a substantially constant axial thickness. Preferably the bag 4 is formed so that it can be filled with the liquid at least substantially free of air or residual gas bubbles, in particular by applying a vacuum to the interior of the bag before the filling operation. In order to permit or assist this filling of the bag at least substantially free of air or residual gas bubbles, the flange 33 can according to a particularly preferred variant taper conically outwards or have an axial thickness decreasing to the outer edge, preferably a thickness decreasing to a minimum value. However, other structural solutions are also possible in this case.

Preferably the connecting piece 28 is provided with at least one valve, in this case the inlet and first non-return valve 13.

The connecting piece 28 can if necessary also form the pump space 12 and/or the cylinder 11 or a cylinder structural part 26 or 27, or the like. Here, various structural solutions are possible. According to one variant the valve 13 accommodated by the connecting piece 28 is set back in the connecting channel 34 with respect to a central through hole of the connecting piece 28 in such a way that the piston 10 can move past at the valve 13 in the hole of the connecting piece 28, so that the pump space 12 can also be formed there.

The bag 4 and the film pieces 29, 30 are preferably formed so as to be flexible. In particular a corresponding film or other material is used to produce and form the film pieces 29, 30.

The bag 4 and the film material is preferably not only liquid-tight but also substantially gas-tight. In particular a composite material, such as a metallised or laminated film, a multi-layer film structure or the like can also be used for this purpose. All these are understood to be covered by the term “film” and “film material”.

In addition the film can be provided internally with a sealing layer or the like, in order to allow and facilitate a laminating or sealing in the joining region 31 and/or joining region 32.

FIG. 10 shows that the joining region 31 and the joining region 32 are in each case formed preferably annularly and all the way round.

The proposed nebuliser 1 is very easy to operate and is very simple in construction.

To use the nebuliser 1 only two steps are necessary. First of all the cap 3 must be opened. The nebulising is then triggered by actuating the control valve 9 (assuming the piston 10 is already in the initial position). The operation is very simple since the energy supply is integrated through the pressure container 8 with the working medium 7, for example compressed air. The control valve is then reset again to the non-actuated position and the cap 3 is closed, wherein the resetting of the control valve 9 can if necessary also take place automatically or after releasing the handle 22 or by closing the cap 3.

The nebuliser 1 is very reliable in operation and in particular allows a complete dispensing of a dose of the liquid. In fact, it is preferably designed so that on actuating the control valve 9 a full pressure and nebulising stroke of the piston 10 always takes place. On account of the internal energy supply an incomplete actuation or faulty operation is excluded.

The proposed nebuliser 1 can preferably be driven pneumatically. This allows a very simple design and construction of the nebuliser 1.

The liquid is contained in the reservoir or bag 4 preferably not under pressure, although this is in principle possible. The pressure generation preferably takes place—at least primarily—through the piston pump and conveying device 5.

The working medium 7 is preferably always separate from the liquid and in particular is not discharged together with this, although this is in principle possible.

The liquid is stored and metered in a very compact and constricted volume. This allows a very compact structure of the nebuliser 1.

In addition a very simple construction of the equipment is possible since a minimum number of structural elements are required, as the nebuliser 1 can be constructed substantially or completely of plastics parts and/or since the tolerance requirements are low. The reservoir is for example formed only of two film pieces 29, 30 and is correspondingly simple and inexpensive to produce.

The cap 3 is preferably designed as a rotatable protective cap. The cap 3 protects in particular a nozzle head and the discharge nozzle 6 against dust or other contamination.

The nebuliser 1 preferably has a mouthpiece 35, shown only by broken lines in FIG. 1, so that a user (not shown) can place the mouthpiece 35 in the mouth for inhaling. After actuating the control valve 9 and initiating the nebulising operation, a nebulising and dispensing of the liquid directly into the user's mouth then takes place. However, in principle other applications and uses of the nebuliser 1 are also possible.

The nebuliser 1 is preferably designed for the brief nebulising of the medicament preparation 2, for example for one to two patient inspirations. However, it can be designed and used also for a longer or continuous nebulising.

The proposed nebuliser 1 preferably operates in particular purely mechanically and/or pneumatically. However, the nebuliser 1 can in principle also operate in any other way and manner.

In contrast to fixed appliances or the like, the proposed nebuliser 1 is preferably designed so as to be transportable, and in particular is a portable hand-held device.

On account of its preferably cylindrical shape and/or its handy size of in particular less than 15 cm in length and/or less than 5 cm in diameter or width, the nebuliser 1 can be carried at all times by a user.

The nebuliser 1 is preferably constructed so that, starting from the discharge end, it first of all comprises the nozzle head and the discharge nozzle 6 and the reservoir. This is followed by the conveying device 5 and the pump. The energy storage device and pressure container 8 then come next. However, in principle other arrangements are also possible.

The nebuliser 1 preferably has a common housing 36 for accommodating the aforementioned components.

Preferably the reservoir together with the inlet and outlet valves or the first and second non-return valves 13, 14 and/or the nozzle head and the discharge nozzle 6 form a first structural group, which is preassembled.

The conveying device 5 preferably forms a second structural group, which is likewise preassembled. For the final assembly the two structural groups are installed in the housing 36.

Preferably the pressure container 8 can subsequently—for example at the time the reservoir 4 is filled—be used with or connected to the liquid or medicament preparation 2. Optionally the connection of the pressure container 8 can also take place later.

If necessary the pressure container 8 can be formed directly by a lower part of the housing 36 or can form the housing 36. It can in this connection be tubular, in the shape of a bottle, or formed flat like a can or disc-shaped.

FIG. 11 shows in a very schematic representation, substantially corresponding to FIG. 2, one variant of the nebuliser 1. Here the pressure container 8 forms at least a (lower) part of the housing 36 of the nebuliser 1. This also constitutes an optionally independently realisable aspect of the present invention.

Particularly preferably the pressure container 8 can also be screwed onto the nebuliser 1 and the housing 36 from below in the manner of a cartridge, the nebuliser 1 then—before the first use—being ready for use. This prevents pressure losses on prolonged storage.

The preferably at least partially elastic design of the housing 36 of the pressure container 8 can minimise the pressure drop also in the case of multiple actuation and use of the nebuliser 1.

The variant according to FIG. 11 shows a preferably partially elastic design of the pressure container 8 and of the outer wall of the nebuliser 1. The preferably corrugated container section 38 is elastically pretensioned when the pressure container 8 is full, so that the pressure drop also with multiple actuation and use of the nebuliser 1 is preferably significantly reduced compared to an inelastic pressure container 8.

The space in which the spring 15 is accommodated and which on the one hand is sealed from the working section 17 in the cylinder 11a and on the other hand from the pump section 16 in the cylinder 11 by the seal 24, is preferably vented outwardly. In the variant illustrated in FIG. 11 the nebuliser 1 and the conveying device 5 preferably comprise a ventilation opening 39 for this purpose. However, other structural solutions are also possible.

It should be noted that individual features and aspects of the variant illustrated in FIG. 11 can also be combined with one another as desired together with the embodiments illustrated in FIGS. 1 to 10.

The filling of the reservoir and bag 4 with the liquid preferably takes place only after assembly of the nebuliser 1, preferably only with a medicament filling device. A suitable labelling, packaging and the like can also be carried out at this time.

The nebuliser 1 is preferably designed so that it can be sterilised before the filling procedure, for example by irradiation. For this purpose the nebuliser 1 is preferably produced at least largely or wholly of a suitable plastics material or the like.

Particularly preferably the reservoir and the bag 4 is filled or can be filled with the liquid free of air or residual gas bubbles.

To fill the reservoir and the bag 4, preferably an external pressure, for example compressed air, is first of all applied in order to collapse the bag 4 completely and expel residual air.

For the actual filling of the reservoir and bag 4 the associated valve 13—and, if the installation and assembly of the nebuliser 1 has already taken place, possibly also the second valve 14—are particularly preferably opened magnetically. This is carried out in particular by attaching a filling machine (not shown) and a magnet or two magnets, which can be integrated in a filling head of the filling machine.

Preferably the valve 13 or the valves 13 and 14 has/have a metallic and/or in particular magnetic or magnetisable valve element 37, particularly preferably in the form of a rounded valve, in order to permit a magnetic opening of the valve 13 or of the valves 13, 14—in particular against the force of a closing spring—to allow liquid to flow in the opposite direction to the dispensing direction. In the valve element 37 or some other element of the valve 13 or 14, a magnetic element or a plurality of magnetic particles can also be injected into the plastics material. These aspects of the magnetic opening and injection of magnetic elements can also be realised independently of one another and also independently of the present invention with other nebulisers 1 or the like.

The valve 13 and/or 14 can however for example also be a magnetic valve or electromagnetically operating valve, which can for example be opened electrically for the filling of the bag 4.

After the filling of the bag 4 in the assembled nebuliser 1, the nozzle head and the discharge nozzle 6 can if necessary be covered by a protective closure, for example a self-adhesive protective film or the like. This can then be removed before the nebuliser is used for the first time.

The proposed nebuliser 1 can if necessary be used not only for nebulising the liquid but optionally also for the spray-like dispensing or other form of dispensing of the liquid.

### LIST OF REFERENCE NUMERALS

1 Nebuliser
2 Medicament preparation
3 Cap
4 Bag
5 Conveying device
6 Discharge nozzle
7 Working medium
8 Pressure container
9 Control valve
10 Piston
11 Cylinder
11a Cylinder
12 Pump space
13 First non-return valve
14 Second non-return valve
15 Spring
16 Pump section
17 Working section
18 Working space
19 Valve element
20 Ventilation channel
21 Connecting channel
22 Handle
23 Spray mist
24 Roll or hose seal
25 End region
26 Cylinder structural part
27 Cylinder structural part
28 Connecting piece
29 Film piece
30 Film piece
31 Joining region
32 Joining region
33 Annular flange
34 Connecting channel
35 Mouthpiece
36 Housing
37 Valve element
38 Container section
39 Ventilation opening

1. A nebuliser (1) for nebulising a liquid medicament preparation (2), with a conveying device (5) in order to presssurise the liquid and nebulise the liquid through a discharge nozzle (6),
characterised in that
the conveying device (5) comprises a piston (10) displaceable in a cylinder (11) and an associated roll or hose seal (24) to seal between the cylinder (11) and the piston (10), and/or
that the conveying device (5) comprises or forms a pump that can be driven by a hydraulic or pneumatic working medium (7), so as to pressurise the liquid and nebulise the liquid through the discharge nozzle (6), or
that the conveying device (5) can be driven with a hydraulic or pneumatic working medium (7) and comprises a pressure booster so as to raise the pressure acting on the liquid compared to the pressure of the working medium (7).

2. The nebuliser according to claim 1, characterised in that air or another gas is provided as working medium (7), and that the pump comprises a piston (10) that can be driven by the working medium (7), so as to pressurise the liquid to be dispensed and nebulise the said liquid through the discharge nozzle (6), wherein a restoring means, (15), is associated with the piston (10) to reset it or to aspirate liquid in a pump space (12).

3. The nebuliser according to claim 1, characterised in that the nebuliser (1) comprises a pressure container (8) for the working medium (7), wherein in particular the pressure container (8) is formed as an insertable or screw-on cartridge or wherein the pressure container (8) is pretensioned or its wall is at least partially elastically deformable to allow a reversible increase of its volume.

4. The nebuliser according to claim 3, characterised in that the pressure booster is designed to raise the pressure by at least a factor of 5, or more, and that the pressure booster comprises a step piston or double piston as piston (10) for pumping the liquid.

5. A nebuliser (1) for nebulising a liquid medicament preparation (2), with a reservoir for the liquid, wherein the reservoir comprises a bag (4) for storing the liquid,
characterised in that
the bag (4) is formed from two film pieces (29, 30) joined to one another in a surrounding or annular joining region (31), or
that the bag (4) is formed lobe-shaped, annular-shaped or disc-shaped and is free of gussets.

6. The nebuliser according to claim 5, characterised in that the joining region (31) is formed at least substantially flat, or that the film pieces (29) are joined to one another on their insides or that the film pieces (29, 30) are joined to one another by bonding, sealing or welding, or that at least one film piece (29, 30) is formed at least substantially disc-shaped or annular-shaped.

7. The nebuliser according to claim 5, characterised in that the nebuliser (1) or the reservoir comprises a connecting piece (8) that is joined to the bag (4) in a joining region (32).

8. The nebuliser according to claim 7, characterised in that the joining region (32) is formed all the way round or annularly, or that the joining region (32) is formed at least substantially flat, or that the connecting piece (28) is joined to a film piece (29, 30), wherein the connecting piece (28) is inserted in an at least substantially flat surface region of the film piece (29, 30).

9. The nebuliser according to claim 7, characterised in that the connecting piece (28) comprises two separate joining regions (32), which are joined to two different film pieces (29, 30) or sides of the bag (4), wherein the joining regions (32) are arranged on opposite sides of the connecting piece (28) or point away from one another, or wherein the joining regions (32) are arranged coaxially or above one another.

10. The nebuliser according to claim 7, characterised in that the connecting piece (28) comprises a flange, (23), to form a joining region (32) or two joining regions (32) for the bag (4), wherein the connecting piece (28) comprises a connecting channel (34) for the fluid connection of the bag (4), wherein the connecting channel (34) extends radially through the flange.

11. The nebuliser according to claim 7, characterised in that the connecting piece (28) is joined to the bag (4) or film pieces (29, 30) of the bag (4) by bonding, sealing or welding or that the connecting piece (28) comprises at least one valve (13).

12. The nebuliser according to claim 5, characterised in that the reservoir is filled or can be filled with the liquid free of air or residual gas bubbles.

13. A nebuliser (1) for nebulising a liquid medicament preparation (2), with a conveying device (5) in order to aspirate the liquid from a reservoir to pressurise the liquid and nebulise the liquid through a discharge nozzle (6), and with a mechanically operating non-return valve (13, 14) that opens under a corresponding pressure or aspiration in order to dispense the liquid in the discharge direction and automatically closes in the opposite direction,
characterised in that
the non-return valve (13, 14) to allow the flow of liquid can be magnetically or electrically opened in the opposite direction to the dispensing direction for filling the reservoir.

14. The nebuliser according to claim 13, characterised in that the non-return valve (13, 14) comprises a metallic valve element (37).

15. (canceled)
